(19) United States
(12) Patent Application Publication
(10) Pub. No.: US 2014/0052004 A1
(43) Pub. Date: Feb. 20, 2014

(54) ENDOSCOPIC CAMERA ILLUMINATION SYSTEM AND METHOD

(71) Applicants: David D'Alfonso, Gaviota, CA (US); Bruce Laurence Kennedy, Santa Barbara, CA (US); Craig Speier, Santa Barbara, CA (US)

(72) Inventors: David D'Alfonso, Gaviota, CA (US); Bruce Laurence Kennedy, Santa Barbara, CA (US); Craig Speier, Santa Barbara, CA (US)

(73) Assignee: ARTHREX, INC., Naples, FL (US)

(21) Appl. No.: 13/586,739

(22) Filed: Aug. 15, 2012

(51) Int. Cl. A61B 1/045; A61B 1/07; A61B 6/00; A61B 1/06

(52) U.S. Cl. 600/476; 600/109

(57) ABSTRACT

An endoscopic camera system having an imaging device for taking images during an exposure period at a predetermined number of frames per second; an illumination system for providing light for the imaging device; and an illumination controller that supplies a drive current to the illumination system, the illumination controller altering the drive current to the illumination system so light is only provided for a portion of each image frame corresponding to the exposure period and for a second portion of each frame outside of the exposure period.

### BACKGROUND OF THE INVENTION

This invention relates generally to endoscopic camera systems and, more particularly, to illumination systems for endoscopic camera systems.

Endoscopic camera systems are used for seeing inside the body of a patient. A typical endoscopic camera system has an illumination system for illuminating the inside of a body cavity and a camera for capturing images of that cavity. Endoscopic camera systems are known in which white light emitting diodes (“LED”) are used as light sources. Light from the white LED may be passed to distal tip of the endoscope using an optical fiber or other waveguide.

However, the output of a white LED is difficult to efficiently couple to an optical fiber or other waveguide. Consequently, to achieve adequate light at the distal tip of the endoscope, input power must be substantial. The generation of substantial input power causes the generation of heat which must be dissipated and which often makes an endoscope, and the user's hand touching it, excessively hot. Moreover, the substantial power required negatively impacts the size and portability of the endoscope because mains power or substantial battery resources are required. If the optical coupling of the LED to an optical fiber or other waveguide is eliminated by mounting the white LED at the distal tip of the endoscope, the heat from the LED itself raises the tip temperature, and the temperature of patient tissue at the operative site, to unacceptable levels.

Additionally, illumination systems are known wherein illumination light is produced by mixing excitation light from an excitation light source, such as an LED or a Laser Emitting Diode, and fluorescent light from phosphors excited by the excitation light. However, unacceptable levels of heat are often generated from the excitation light source. Moreover, substantial power is still required for the excitation light source.

Accordingly, a need exists for an improved illumination system for an endoscope that remedies the shortcomings of the prior art.

### SUMMARY OF INVENTION

Accordingly, the present invention is directed to an endoscopic camera system having a system for dynamically adjusting the illumination to the lowest level needed to maintain image quality.

An endoscopic camera system according to an embodiment of the present invention has an imaging device for taking images during an exposure period at a predetermined number of frames per second. The endoscopic camera system also has an illumination system for providing light for the imaging device. The endoscopic camera system further has an illumination controller that supplies a drive current to the illumination system, the illumination controller altering the drive current to the illumination system so that light is only provided for a portion of each frame corresponding to the exposure period and for a second separate portion of each frame outside of the exposure period.

The illumination controller may only provide drive current for a portion of the exposure period to control image exposure. Additionally, color of light from the illumination system may change with changes to the supply of drive current to the illumination system and the system may have an image processor that adjusts images to compensate for drive current based changes in light color.

In an additional embodiment, the light source has a light source for emitting light of a first wavelength band; phosphors excited by the light of the first wavelength band to emit light of a second wavelength band; and a light guide optically coupling the light source to the phosphors. Optionally, the light source is a laser diode emitting blue light. Additionally, the phosphors may include a plurality of different phosphors emitting light of different wavelengths in response to light from the light source.

In an additional embodiment, the present invention is directed an endoscopic camera system having a camera head with an imaging device for taking images during an exposure period at a predetermined number of frames per second and an illumination system for providing illumination for the imaging device. The endoscopic camera system also has a camera control unit coupleable to the camera head.

The camera control unit has an illumination controller that supplies drive current to the illumination system, the illumination controller altering the drive current to the illumination system so that the light source only receives the drive current for a portion of each image frame corresponding to the exposure period. The camera control unit also has an image processor that alters image processing based on the amount of drive current received by the light source in a frame. The endoscopic camera system also has a storage device coupled to the image processor for storing drive current based image processing parameters.

Additionally, the camera head may have a housing; and a shaft coupled to the housing, the shaft having a proximal end and a distal end for insertion into an object to be viewed. A light emitting diode emitting an excitation light may be positioned in the housing. A light guide may optically coupled to the light emitting diode and extended through the shaft. A plurality of phosphors may be positioned proximal to the distal end of the shaft and optically coupled to the light guide to receive excitation light from the light emitting diode.

In an additional embodiment, the present invention is directed to a method for controlling an illumination system that has a light source for emitting an excitation light of a first wavelength and phosphors excited by the excitation light to emit light of a second wavelength for use with an imaging device for taking images during an exposure period at a predetermined number of frames per second. The method has the steps of: supplying a first drive current to the light source for the exposure period of the imaging device; and supplying a second drive current to the light source for a separate period of time within each frame outside of the exposure period of the imaging device. The period of the second drive current may be constant.

### BRIEF DESCRIPTION OF THE DRAWINGS

The features, aspects and advantages of the present invention will become better understood with regard to the following description, appended claims and accompanying figures wherein:

FIG. 1 is a schematic drawing of an endoscope system according to a first embodiment of the present invention; and

FIG. 2 is a schematic drawing of a camera head usable in the endoscope system of FIG. 1;

FIG. 3 is a schematic drawing of the distal tip section of the camera head of FIG. 2;

FIG. 4 is a schematic drawing of the distal tip section of a camera head usable in the endoscope system of FIG. 1 according to a first alternative embodiment of the present invention;

FIG. 5 is a schematic drawing of the distal tip section of a camera head usable in the endoscope system of FIG. 1 according to a second alternative embodiment of the present invention;

FIG. 6 is a diagram illustrating methods of altering a drive current to a light source in relation to the exposure period according to embodiments of the present invention;

FIG. 7 is a diagram illustrating a method of altering a drive current to a light source in relation to the exposure period to reduce the appearance of flicker according to an embodiment of the present invention;

FIG. 8 is a diagram illustrating a method of altering a drive current to a light source in relation to a variable exposure period;

FIG. 9 is a diagram illustrating a change in light source color relative to the temperature of the light source;

FIG. 10 is a graph showing the blue and red gain necessary to maintain white balance in relation to changing drive current in an example system according to an embodiment of the present invention; and

FIG. 11 is a graph showing the blue gain necessary to maintain white balance in relation to changing drive current over time in an example system according to an embodiment of the present invention.

### DETAILED DESCRIPTION OF THE PREFERRED EMBODIMENTS

In the following description of the preferred embodiments, reference is made to the accompanying drawings which show by way of illustration specific embodiments in which the invention may be practiced. Wherever possible, the same reference numbers will be used throughout the drawings to refer to the same or like parts. It is to be understood that other embodiments may be utilized and structural and functional changes may be made without departing from the scope of the present invention.

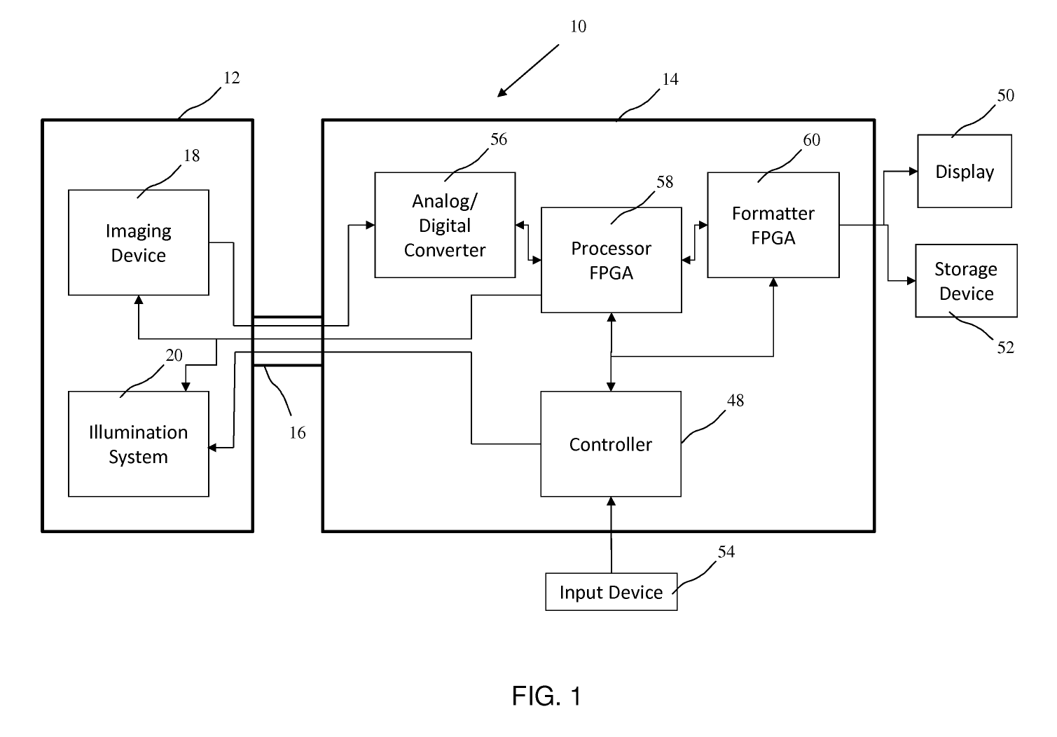

An endoscopic imaging system 10 according to an embodiment of the present invention is shown in FIG. 1. The endoscopic imaging system 10 allows for internal features of a body of a patient to be viewed without the use of traditional, fully invasive surgery. Additionally, the endoscopy system may be used for imaging of hard to reach parts of structures or in other applications where direct optical viewing is compromised.

The endoscopic imaging system 10 has a camera head 12 and a camera control unit 14. In an embodiment, the camera head 12 is coupled to the camera control unit 14 via a cable 16 to facilitate data transfer between the camera head 12 and the camera control unit 14. In an alternative embodiment, the camera head 12 is wirelessly coupled to the camera control unit 14 such as via IEEE 802.11b, or IEEE 802.11n or ultra-wide band (UWB).

The camera head 12 acquires image data and transmits it to camera control unit 14 to process a usable image. The camera head 12 may be used together with an endoscope or other medical instruments for transmitting image data. The camera head 12 may include one or more imaging devices 18, utilizing a variety of technology types. For example, the imaging devices may include one or more charge coupled device (CCD) sensors or complementary metal-oxide-semiconductor (CMOS) sensors. CCD image sensors are classified into a frame transfer type, an interline transfer type, and a frame/interline transfer type depending on the method for reading out signal charges obtained at light-receiving pixels. As will be further explained, a shutter driver controls operation of the imaging device 18.

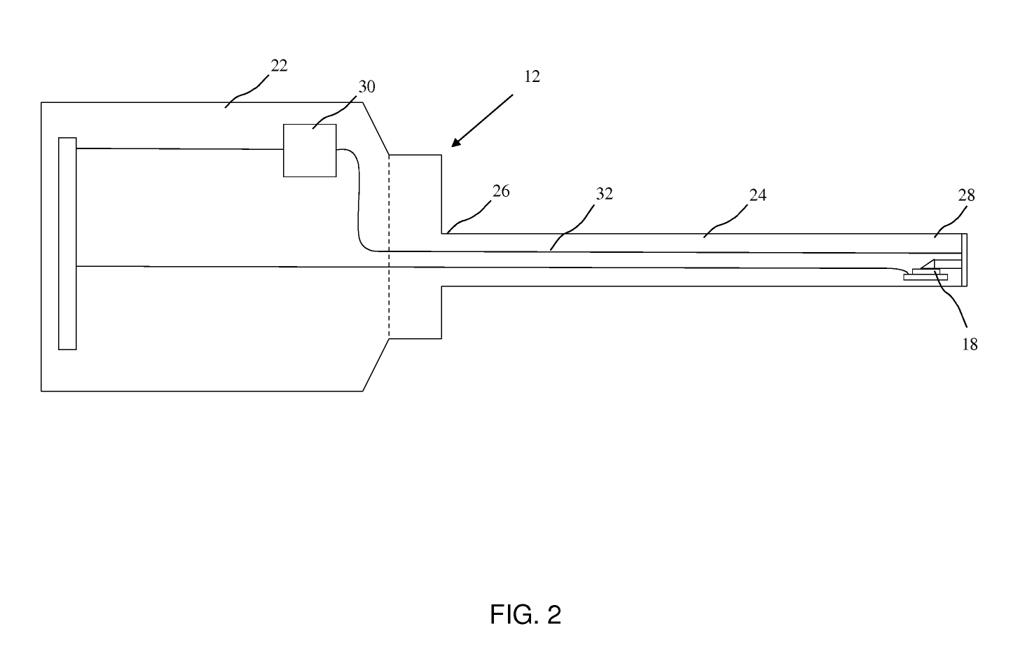

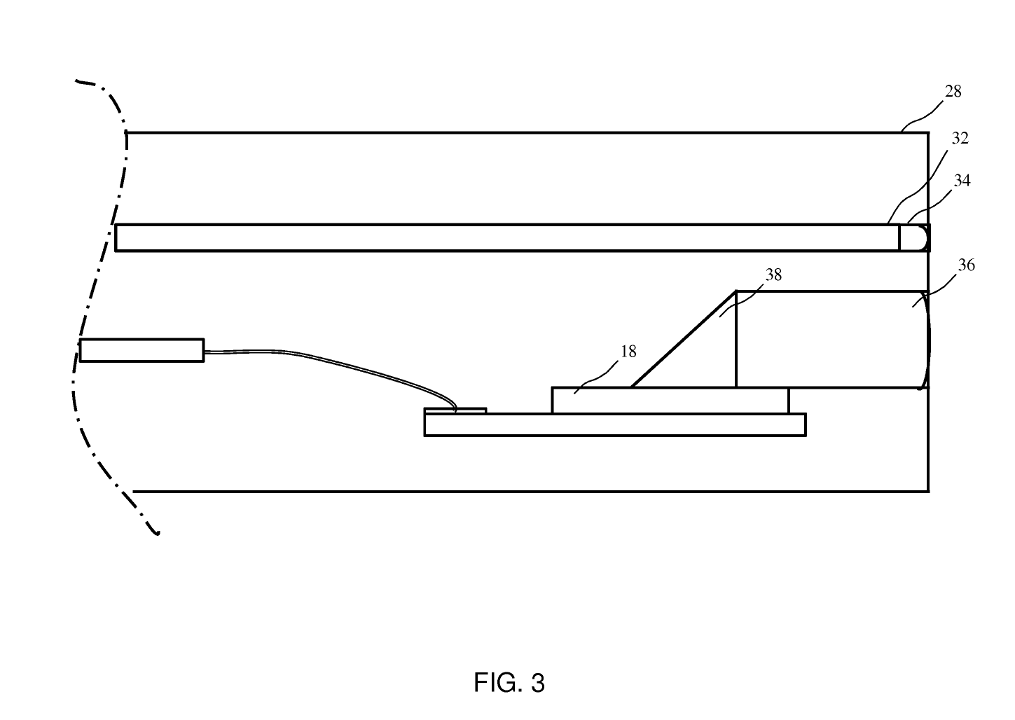

In an embodiment of the present invention, as shown in FIGS. 2 and 3, the camera head 12 has a housing 22 and a shaft 24 coupled to the housing 22. The shaft 24 has a proximal end 26 adjacent to the housing 22 and a distal end 28 for insertion into a body or other area to be viewed. The illumination system includes a light source 30 positioned in the housing 22. Preferably, the light source 30 is at least one white LED. A light guide 32 is optically coupled to the light source 30. As will be appreciated by one of skill in the art, optics such as lenses may be placed between the light source 30 and the light guide 32.

The light guide 32 extends through the shaft 24 to convey light from the light source 30 to near the distal end 28 of the shaft. The light guide 32 is typically formed of optical grade materials, such as acrylic resin, polycarbonate, epoxies and glass. The light guide 32 may terminate near the distal end 28 of the shaft, the exposed end of the light guide polished to pass light out through the distal end of the shaft. Alternatively, near the distal end 28 of the shaft, the light guide 32 may be coupled to a transmission optical assembly 34 that focuses light from the light source and passes the light out through the distal end 28 of the shaft.

Light emitted from the illumination system 20 is applied to an object located outside of the camera head 12. Reflection light comes into the distal end of the shaft 24 and is directed by an imaging optical assembly 36 onto the imaging device 18. In an embodiment, the imaging optical assembly 36 includes a prism 38. Operation of the imaging device 18 is discussed in more detail below.

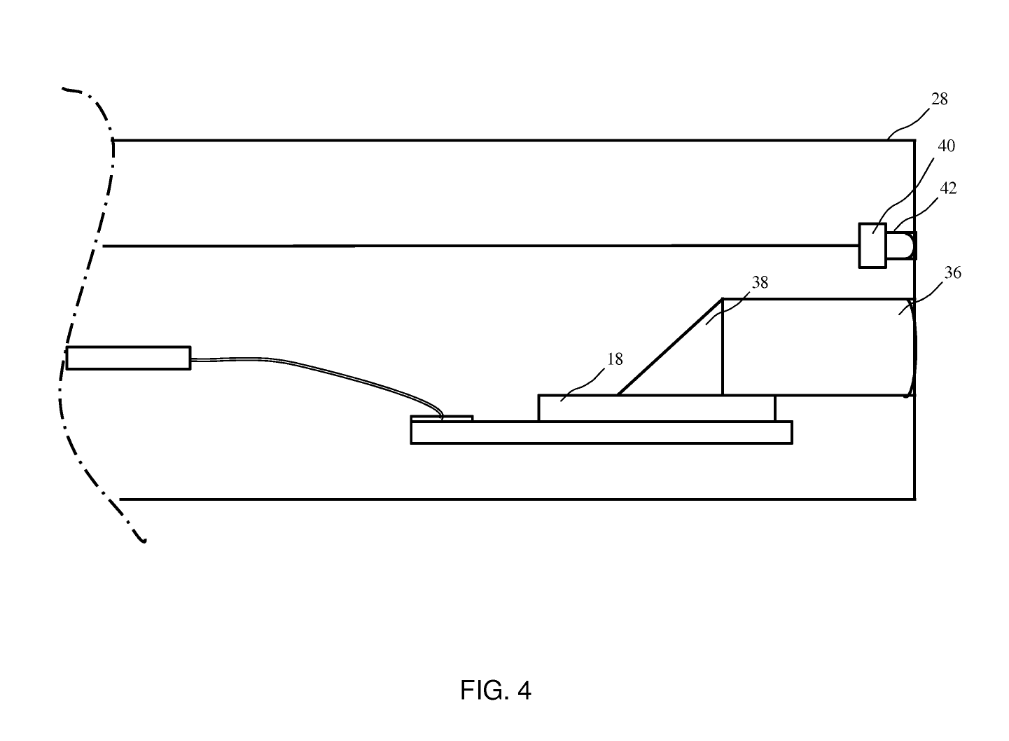

In a first alternative embodiment of the present invention, as shown in FIG. 4, the illumination system 20 includes at least one white LED 40 positioned proximal to the distal end of the shaft instead of in the housing 22. The white LED is 40 is coupled to a transmission optical assembly 42 that focuses light from the white LED and passes the light out through the distal end 28 of the shaft.

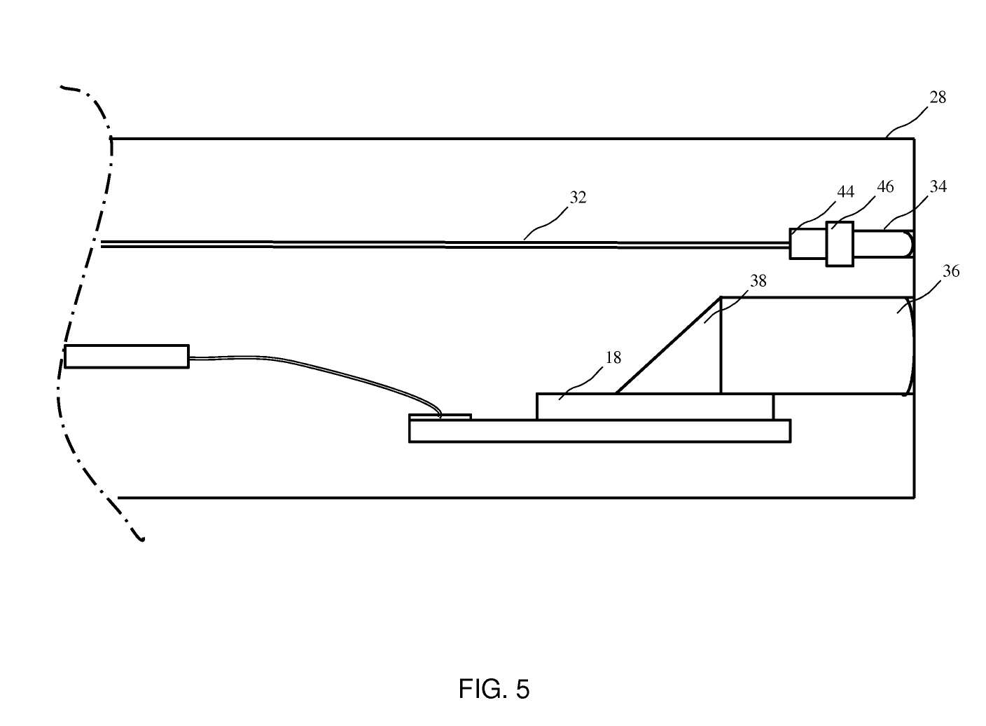

In a second alternative embodiment of the present invention, the light source 30 positioned in the housing 22 is a blue laser emitting diode emitting an excitation light. The light guide 32 is optically coupled to the light source 30, such as via lenses. As shown in FIG. 5, the light guide 32 extends through the shaft 24 to convey excitation light from the light source 30 to near the distal end 28 of the shaft. Near the distal end of the shaft, excitation light from the light guide 32 is directed, via excitation optics 44, onto phosphors 46. The phosphors 46 emit light of a broad wavelength from green to yellow and red. The excitation light is mixed with the light from the phosphors 46 to be converted into light of a wavelength band for white light. As party of the excitation optics 44, a short wavelength (high pass) filter may be placed between the light guide 32 and the phosphors 46 to prevent reflected energy from returning to the light source.

The phosphors 46 may be formed from phosphoric materials into a plate, or a glass kneaded with phosphors. The phosphors 46 may also be coated onto optical glass. The phosphors may include, for example, silicate (orange), garnett (yellow) or aluminate (green) classes. Combinations of two or more classes are typically used to produce broad spectrum (white) light. Light from the phosphors and any residual excitation light is passed through the transmission optical assembly 34 that focuses the light and passes the light out through the distal end 28 of the shaft. Electrical energy from mains power or from a battery powers the light source 32 and can be adjusted to set the level of illumination.

A laser diode is advantageous, because the directed nature of the output is easily coupled into light transmitting fibers. Placing the spectrum shifting phosphor at the endoscope tip produces white light in the same way as commercial white LED's without the heat of the diode itself. This limits the amount of heat transmitted to a patient's tissues proximal the distal end of the shaft 24.

In an additional embodiment of the present invention, a portion of the camera head is designed to be disposable. For example, as shown by the dotted line in FIG. 2, the light source, which may be a laser emitting diode, may be located in the reusable portion of the housing and the shaft 24 and a portion of the housing may be designed to be disposable. This is advantageous in that the relatively expensive parts, such as a laser diode, are part of a reusable part while the relatively inexpensive parts, such a light guide and phosphors are in a separable and disposable part. An efficient optical coupling from the light source, and particularly from a laser emitting diode, to the light guide reduces excitation light loss at the junction between these reusable and the disposable parts of the camera head.

In an additional embodiment of the present invention, the light source is positioned outside of the camera head. Light from the light source is directed to the camera head by a light guide. In an embodiment, the light source is positioned inside the camera control unit and a light guide directs light from the light source in the camera control unit to the camera head. As will be further explained, a light source driver controls power to the light source of the illumination system.

The camera control unit 14 will now be explained in more detail with reference to FIG. 1. The camera control unit 14 is preferably a programmable unit containing sufficient processing capacity to accommodate a wide range of control, user interface and image acquisition/processing functions. The camera control unit 14 has a controller 48 and runs program applications providing for a variety of capabilities. For instance, an image capture and display capability allows for both display of a live feed of an image through a display 50 coupled to the camera control unit 14, as well as image capture. Captured images may be stored to an external storage device 52 coupled to the camera control unit 14 such as via a Universal Serial Bus interface. Alternatively, the external storage device 52 may a storage device accessible via the Internet. Additionally, the controller 48 may receive information and other input from one or more input devices 54.

In an embodiment, analog RGB data is transmitted from the imaging device 18 to the camera control unit 14. The Analog RGB data passes through a digital/analog converter 56 to a processor field programmable gate array (FPGA) 58 where the video is processed. The processed video is then passed to a formatter FPGA 60 where the video is formatted into various display formats. The formatter FPGA 60 may also overlay information, such as patient and/or doctor information, onto the video. The formatted video may be converted back to an analog signal for display. The formatted video is sent to the display 50 and/or the storage device 52.

The camera control unit 14 issues commands to the camera head 12 to adjust its operating characteristics, and the camera head 12 may send confirmation to the camera control unit 14 that it received the commands. The processor FPGA 56 and/or the controller 48 may communicate with a shutter driver either in the camera control unit or the camera head to control the exposure period of the imaging device. Additionally, the processor FPGA 56 and/or the controller may communicate with a light source driver either in the camera control unit or the camera head to control power to the illumination source of the illumination system 20.

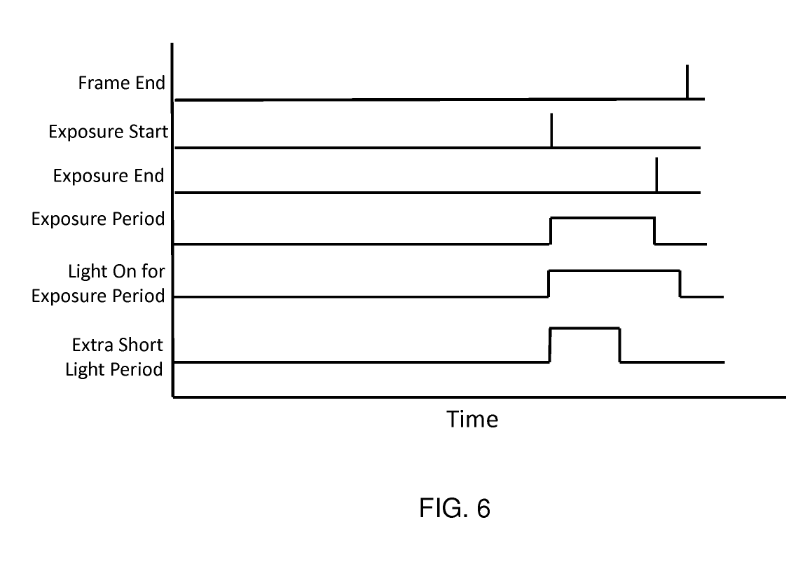

Preferably, power to the light source is controlled in relation to an exposure period of the imaging device. FIG. 6 shows the relationship between the imaging device 18 and the illumination system 20 as a function of time according to various embodiments of the present invention. In FIG. 6, time is on a horizontal axis with approximately one video frame shown. The imaging device 18 captures about 60 frames every second to form the video signal that is processed and ultimately displayed for a user and/or saved to the storage device 52.

During each frame, electrons are accumulated at each pixel of the imaging device 18 starting at an exposure start time and ending at an exposure end time. Both the exposure start time and the exposure end time are adjustable and controlled by an auto exposure system in the camera control unit 14. In an embodiment, the processor FPGA contains the autoexposure system.

As shown in FIG. 6, the exposure period is the time between the exposure start time and the exposure end time. The exposure period is adjusted to correct for under or over exposure during the previous frame with the goal that a high quality image is always displayed. Exposure period adjustments are typically implemented every other frame rather than every frame although exposure adjustments can be made more or less frequently.

In an embodiment, the camera control unit uses the exposure timing signals to provide a driving current to the light source only during the exposure period when the image device 18 is accumulating electrons to generate an image. Any light generated outside of the exposure period is wasted, draws unnecessary power, and leads to undesirable excess heating of the endoscope and/or a patient.

Referring again to FIG. 6 and the line entitled “Light On for Exposure Period”, in an embodiment, the light source driver may be controlled to turn on the light source 30 at the exposure start time and turn off the light source 30 at some point after the exposure end time. In this embodiment, the period of time in which the light source receives power does not need to perfectly match the exposure period, but needs to include the exposure period. As shown by the line entitled “Light On for Exposure Period” in FIG. 6, the light may be turned on at the exposure start time and off at the end of the frame making the exposure light period extend past the exposure end time by a variable amount depending on the position of exposure end time relative to the end of the frame. Alternatively, the light source 30 may be turned on at the exposure start time and turned off at the exposure end time.

In an additional embodiment, as shown in FIG. 6 as the line entitled “Extra Short Light Period”, the light source driver is controlled to turn on the light source 30 at the exposure start time and off at a time prior to the exposure end time, thus providing power to the light source 30 for a period shorter than the exposure period. This has the advantage of reducing the light to less than the minimum exposure time available for control of the imaging device by the shutter driver. The extra short light period may be controlled as a fixed fraction of the camera exposure period unlike an exposure light pulse that ends at a fixed time after the exposure end time. Also, with the use of a synchronized light source, it is possible to set the imaging device to expose for the full frame time and use the light source driver and light source to control exposure.

### Flicker Reduction

As explained above, it is desirable to turn on and off a light source within each image frame to reduce power consumption and heat generation. At a 60 Hz frame rate, the light source is on for some fraction of each 1/60 of a second which amounts to a variable duty cycle train of light flashes at the rate of 60 per second. The camera does not see these flashes because the exposure period is typically synchronized to times at which the light source is energized.

However a human viewer observing the light source directly at the operative site may see annoying flashes. This is because the flash rate is at the flicker fusion flash threshold of the human visual system. Unfortunately, the visual system sensitivity to flicker is greatest in peripheral vision and may pose a distraction to a surgeon or other endoscope operator. This is especially a problem with some light guides that allow small amounts of light to escape through their translucent jacket material as an indication that the light source is operating.

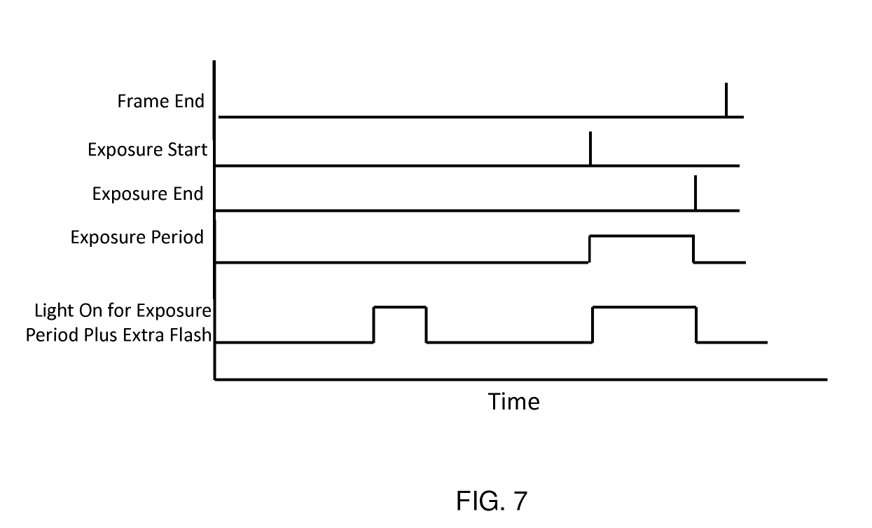

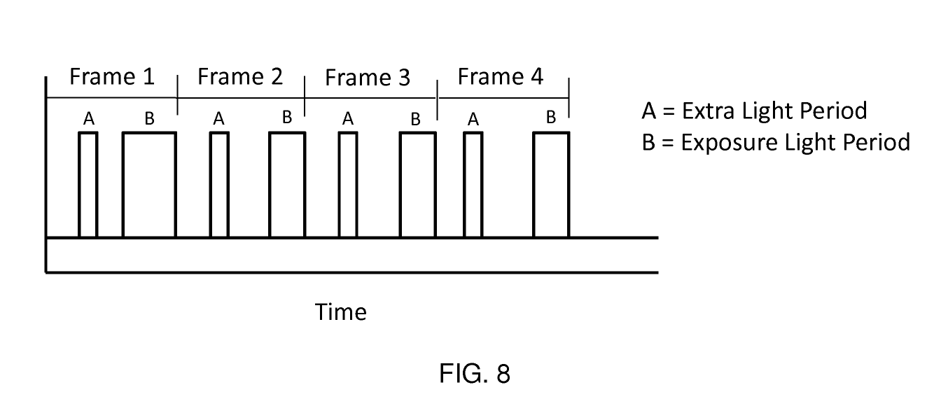

As illustrated in FIG. 7, to suppress any perception of flicker, an additional short duration pulse of light may be added between exposure light pulses. In a preferred embodiment, the extra light period is added in the middle of the period between exposure light periods, thus doubling the flash rate and exceeding the fusion flash threshold. Preferably, as shown illustrated in FIG. 8, the extra light period is of a fixed duration, which is minimized to preserve the heat and power reducing purpose of variably driving the illumination source in relation to the exposure period, while the exposure light period is variable to correspond with a variable exposure period. The duration of the extra light period may be modified based on the factors that influence the flash fusion threshold, such as flash intensity and background lighting.

In an additional embodiment of the present invention, the light source may be dimmed, rather than completely turned off for a portion of each frame, such as outside of the exposure period, to reduce heat and power consumption. In an additional embodiment where multiple LED's are used for the illumination source, a subset of the LED's may be turned off or dimmed for a portion of each frame to reduce heat and power consumption. In an additional embodiment, there may be more than one extra light period outside of the exposure period to reduce flicker and reduce heat and power consumption.

### Adapting to Illumination Source Color Temperature Shift

Many light sources, such as arc lamps, halogen lamps and LEDs change color over a fairly wide range if current alone is used to change output level. In the case of an LED light source, as the output is reduced by adjusting the ratio of on to off periods within a frame time, the temperature of the emitting LED chip decreases. The change in temperature shifts the color temperature of the white light output.

Additionally, in an illumination system utilizing an excitation light source, such as a blue laser emitting diode, to excite light from phosphors, temperature based variation in the excitation light causes a change in efficiency of light emission from the phosphors, which results in changing volume or chromaticity of the finally produced white light. Color temperature shift in the illumination system is undesirable, because it changes the color of the image displayed by the endoscopic camera system. Color changes to the image displayed by the endoscopic camera system, may make it is difficult or impossible to make accurate systematic diagnoses.

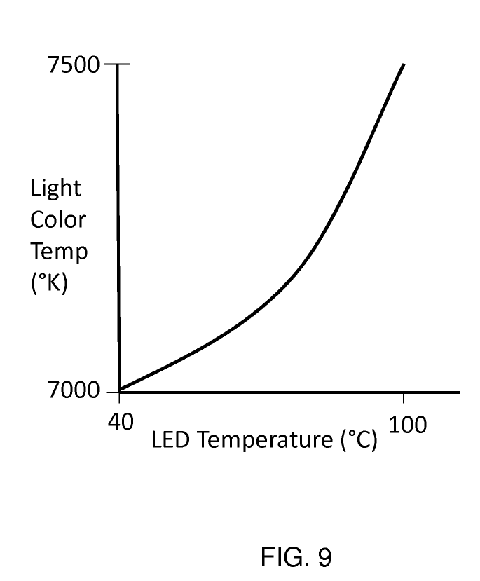

To correct color temperature shift, the processor FPGA 58 can compensate by adjusting the color balance of the video signal in the camera image processing system. There is a relationship between the LED temperature and the amount of color temperature shift. As shown in FIG. 9, as the temperature of the LED chip decreases, the color temperature of light emitted from the LED tends to change from bluish (high color temperature) to reddish (low color temperature). As LED power is changed by adjusting the drive signal, the processor FPGA can adjust color balance to hold the output image color temperature stable over the expected operating range.

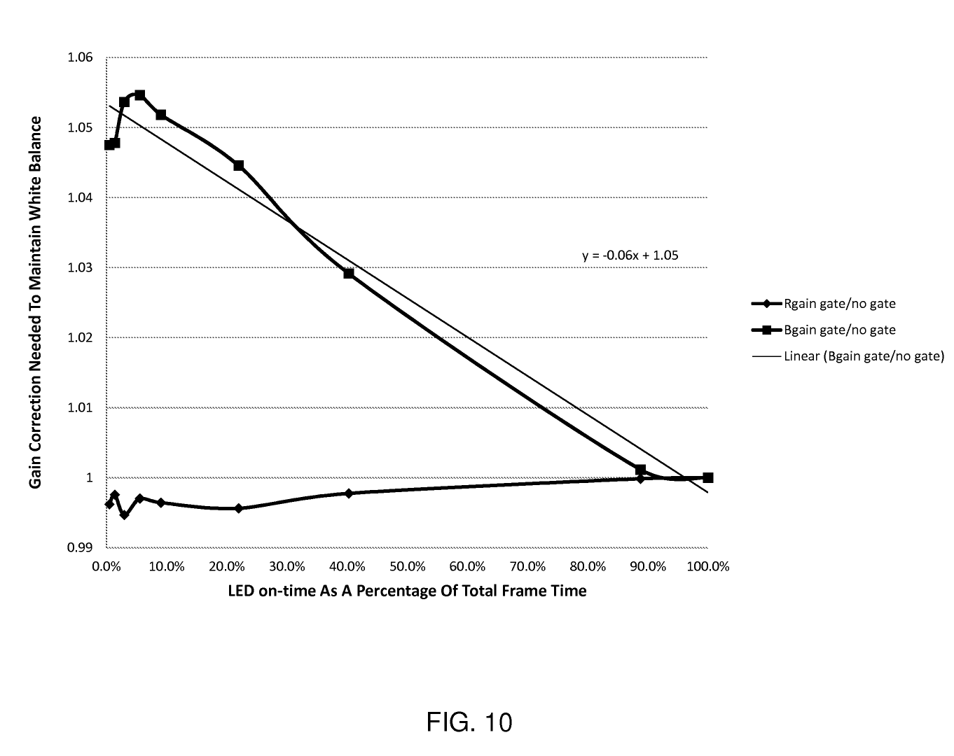

There is a known relationship between an on time duty cycle of the LED and the necessary color balance required. FIG. 10 shows the red and blue gain required to maintain white balance in an example system in which the LED on-time as a percentage of total frame time was varied. As seen from FIG. 10, in the example system, there was little need for red correction, while blue correction was substantially linear in response to changes in the LED on-time. The same red and blue gain controls now used for routine white balance may be adjusted in proportion to the LED pulse width as part of the automatic exposure system to compensate for LED color shift stemming from changes in the LED drive signal. There is also a known relationship between an LED chip temperature and the on time duty cycle of the LED and the color balance may be adjusted based on measured or projected LED temperature changes. The correction information may be stored in a memory, such as in a database, accessible to the processor FPGA for use by the processor FPGA in color balance.

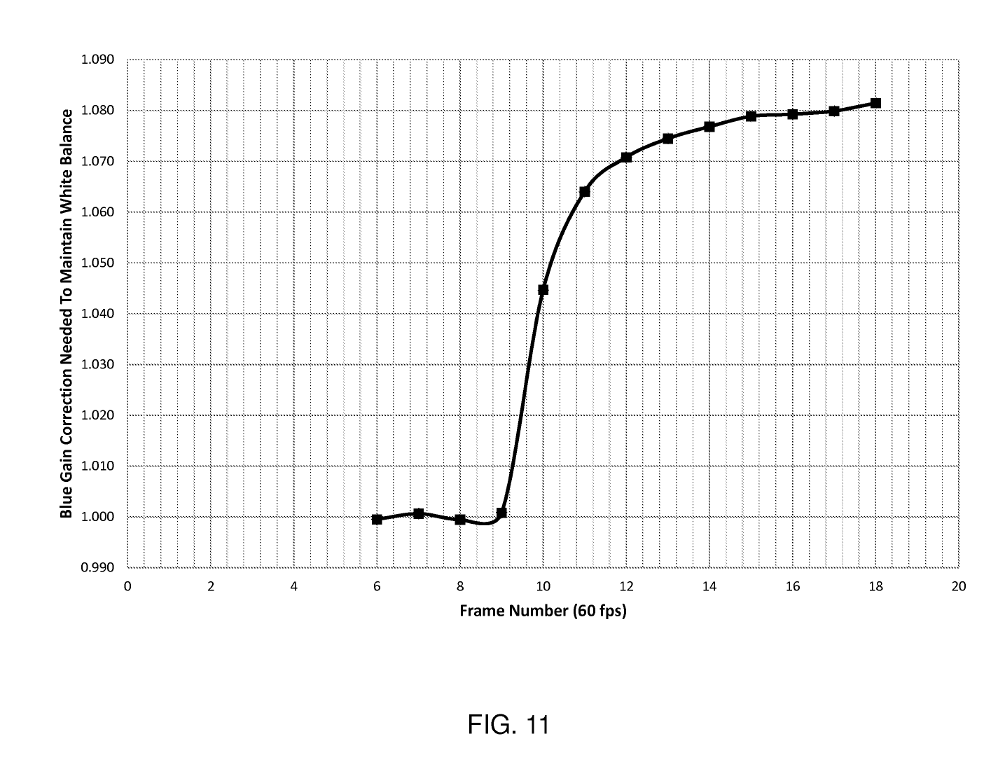

The pulse signal to the light source may change every frame and the color balance adjustments in the video processing can also be made to change every frame to match the expected color shift. FIG. 11 shows the blue correction necessary over time in an example system wherein the drive signal to the LED was changed from being constant (on for the full frame) to being pulsed at a width equal to an exposure period occupying about 3% of the frame time. The change in LED drive signal occurs after frame 9. As shown in FIG. 11, the color correction needed to compensate typically lags somewhat in time behind changes in drive power (duty cycle). The color correction needed typically exhibits a first order or simple low pass filter response. The time lag may be factored into the color correction to keep the final camera image color stable.

There is disclosed in the above description and the drawings, an endoscope illumination system and method which overcomes the disadvantages associated with the prior art. However, it will be apparent that variations and modifications of the disclosed embodiments may be made without departing from the principles of the invention. The presentation of the preferred embodiments herein is offered by way of example only and not limitation, with a true scope and spirit of the invention being indicated by the following claims.

Any element in a claim that does not explicitly state “means” for performing a specified function or “step” for performing a specified function, should not be interpreted as a “means” or “step” clause as specified in 35 U.S.C. §112.

What is claimed is:

1. An endoscopic camera system comprising:
an imaging device for taking images during an exposure period at a predetermined number of frames per second;
an illumination system for providing light for the imaging device; and
an illumination controller that supplies a drive current to the illumination system, the illumination controller altering the drive current to the illumination system so that light is only provided for a portion of each frame corresponding to the exposure period and for a second separate portion of each frame outside of the exposure period.

2. The endoscopic camera system of claim 1 wherein the illumination controller provides drive current for a portion of the exposure period to control image exposure.

3. The endoscopic camera system of claim 1 further comprising an image processor;
wherein a color of the light from the illumination system changes with changes to the supply of drive current to the illumination system; and
the image processor adjusts the images to compensate for drive current based changes in light color.

4. The endoscopic camera system of claim 1 wherein the light source further comprises:
a light source for emitting light of a first wavelength band;
phosphors excited by the light of the first wavelength band to emit light of a second wavelength band; and
a light guide optically coupling the light source to the phosphors.

5. The endoscopic camera system of claim 4 wherein the light source is a laser diode emitting blue light.

6. The endoscopic camera system of claim 5 wherein the phosphors comprises a plurality of different phosphors emitting light of different wavelengths in response to light from the light source.

7. An endoscopic camera system comprising:
a camera head further comprising:
an imaging device for taking images during an exposure period at a predetermined number of frames per second;
an illumination system for providing illumination for the imaging device; and
a camera control unit coupleable to the camera head, the camera control unit further comprising:
an illumination controller that supplies drive current to the illumination system, the illumination controller altering the drive current to the illumination system so that the light source only receives the drive current for a portion of each image frame corresponding to the exposure period;
an image processor that alters image processing based on the amount of drive current received by the light source in a frame; and
a storage device coupled to the image processor, the storage device storing drive current based image processing parameters.

8. The endoscopic camera system of claim 7 wherein the camera head further comprises:
a housing; and
a shaft coupled to the housing, the shaft having a proximal end and a distal end for insertion into an object to be viewed.

9. The endoscopic camera system of claim 8 wherein the camera head further comprises a light emitting diode emitting an excitation light positioned in the housing;
a light guide optically coupled to the light emitting diode and extending through the shaft;
a plurality of phosphors positioned proximal to the distal end of the shaft and optically coupled to the light guide to receive excitation light from the light emitting diode.

10. A method for controlling an illumination system that has a light source for emitting an excitation light of a first wavelength and phosphors excited by the excitation light to emit light of a second wavelength for use with an imaging device for taking images during an exposure period at a predetermined number of frames per second, the method comprising the steps of:
supplying a first drive current to the light source for the exposure period of the imaging device; and
supplying a second drive current to the light source for a separate period of time within each frame outside of the exposure period of the imaging device.

11. The method of claim 10 wherein the period of the second drive current is constant.
